(19) United States
(12) Patent Application Publication
(10) Pub. No.: US 2017/0368351 A1
(43) Pub. Date: Dec. 28, 2017

(54) RETINAL PROSTHESIS

(71) Applicant: NANO-RETINA, INC., Wilmington, DE (US)

(72) Inventor: Tuvia LIRAN, Qiryat Tivon (IL)

(73) Assignee: NANO-RETINA, INC., Wilmington, DE (US)

(21) Appl. No.: 15/195,212

(22) Filed: Jun. 28, 2016

(51) Int. Cl. A61N 1/36; A61N 1/378; A61N 1/05

(52) U.S. Cl. CPC A61N 1/36046; A61N 1/0543; A61N 1/3787

(57) ABSTRACT

Intraocular apparatus is provided for be implantation entirely in a subject's eye. The intraocular apparatus includes a photosensor array including a plurality of photosensors configured to receive an ambient image, and a power source, for powering the apparatus. The intraocular apparatus additionally including a flexible 0.4-3 mm electrical connector, connecting the photosensor array to the power source. Other applications are also described.

### FIELD OF THE INVENTION

The present invention relates generally to implantable medical devices, and specifically to a retinal prosthesis.

### BACKGROUND

Retinal malfunction, due to degenerative retinal diseases, is a leading cause of blindness and visual impairment. Implantation of a retinal prosthesis is a technology for restoring some useful vision in individuals suffering from retina-related blindness.

The retina is a multi-layered light-sensitive structure that lines the posterior, inner part of the eye. The retina contains photoreceptor cells, rods and cones, which capture light and convert light signals into neural signals transmitted through the optic nerve to the brain.

### SUMMARY OF THE INVENTION

In accordance with some applications of the present invention, intraocular apparatus is provided for implantation entirely in a subject's eye. The intraocular apparatus is typically implanted for stimulation of the retina of the subject suffering from a retinal disease, in order to restore at least partial vision in the subject. The intraocular apparatus typically comprises a photosensor array comprising a plurality of photosensors configured to receive an ambient image, a power source configured to power the apparatus, and a flexible 0.4-3 mm electrical connector, connecting the photosensor array to the power source.

Typically, the apparatus further comprises an electrode array comprising electrodes, and driving circuitry coupled to the power source and to the photosensor array, and configured to receive power from the power source to drive the electrodes to apply current pulses to the retina in response to signals from the photosensor array, in order to stimulate the retina.

In accordance with some applications of the present invention, the power source comprises at least one energy receiver configured to receive non-visible light, typically infra-red light, through the lens of the eye and to extract power from the non-visible light. Typically, the at least one energy receiver comprises at least first and second energy receivers, positioned on either side of the photosensor array, and the flexible electrical connector is a first flexible electrical connector and connects the first energy receiver to the photosensor array, and the intraocular apparatus further comprises a second flexible electrical connector, which connects the second energy receiver to the photosensor array.

Additionally or alternatively, the power source comprises at least one power storage element, for example, a rechargeable battery configured to power the intraocular apparatus when the intraocular apparatus is not receiving energy from outside of the eye. For some applications, the power storage element stores sufficient charge for operation of the intraocular apparatus for 1-10 hours.

Typically, the at least one power storage element comprises first and second power storage elements, positioned on either side of the photosensor array, and the flexible electrical connector is a first flexible electrical connector and connects the first power storage element to the photosensor array and the intraocular apparatus further comprises a second flexible electrical connector, which connects the second power storage to the photosensor array.

There is therefore provided in accordance with some applications of the present invention, intraocular apparatus configured to be implanted entirely in a subject's eye, the intraocular apparatus including:

a photosensor array including a plurality of photosensors configured to receive an ambient image;

a power source, configured to power the intraocular apparatus; and

a flexible 0.4-3 mm electrical connector, connecting the photosensor array to the power source.

For some applications, the flexible connector includes thinned silicon.

For some applications, the photosensor array, the power source and the flexible electrical connector are formed along a single piece of thinned silicon.

For some applications, the power source includes at least one power storage element, configured to power the intraocular apparatus when the intraocular apparatus is not receiving energy from outside of the eye.

For some applications, the at least one power storage element includes a rechargeable battery configured to store sufficient charge for operation of the intraocular apparatus for 1-10 hours.

For some applications, the rechargeable battery has a total charge of 0.1-10 mAh.

For some applications, the at least one power storage element includes a plurality of stacked dies, each die including a plurality of miniature batteries integrated into the die.

For some applications, the at least one power storage element includes first and second power storage elements, positioned on either side of the photosensor array,

the flexible electrical connector is a first flexible electrical connector and connects the first power storage element to the photosensor array, and

the intraocular apparatus further includes a second flexible electrical connector, which connects the second power storage element to the photosensor array.

For some applications, the power source includes at least one energy receiver configured to receive non-visible light through the lens of the eye and to extract power from the non-visible light.

For some applications, the at least one energy receiver is configured to receive light with wavelength that is outside of 390-700 nm.

For some applications, the at least one energy receiver includes first and second energy receivers, positioned on either side of the photosensor array,

the flexible electrical connector is a first flexible electrical connector and connects the first energy receiver to the photosensor array, and

the intraocular apparatus further includes a second flexible electrical connector, which connects the second energy receiver to the photosensor array.

For some applications, the power source further includes at least one power storage element, configured to power the intraocular apparatus when the intraocular apparatus is not receiving energy from outside of the eye.

For some applications, the apparatus further includes an extraocular device including a light source configured to emit the non-visible light toward the eye, wherein the at least one power storage element is configured to store power extracted from the non-visible light for at least one hour, and wherein the intraocular apparatus is configured to use the stored power to power the intraocular apparatus.

For some applications, the light source includes a laser.

For some applications, the at least one power storage element includes a rechargeable battery configured to receive the power extracted by the energy receiver.

For some applications, the intraocular apparatus further includes (i) an electrode array including electrodes, and (ii) driving circuitry coupled to the power source and to the photosensor array, and configured to receive power from the power source to drive the electrodes to apply currents to the retina in response to signals from the plurality of photosensors in the photosensor array.

There is further provided in accordance with some applications of the present invention, intraocular apparatus configured to be implanted entirely in a subject's eye, the intraocular apparatus including:

a photosensor array including a plurality of photosensors configured to receive an ambient image;

a power source, configured to power the intraocular apparatus; and

a flexible electrical connector, connecting the photosensor array to the power source,

the photosensor array, the power source and the flexible electrical connector being formed along a single piece of thinned silicon.

There is further provided in accordance with some applications of the present invention, intraocular apparatus configured to be implanted entirely in a subject's eye, the intraocular apparatus including:

a photosensor array including a plurality of photosensors, configured to receive an ambient image; and

a flexible power source including thinned silicon and coupled to the plurality of photosensors, configured to power the intraocular apparatus and including a photovoltaic energy receiver configured to receive non-visible light and to extract power from the non-visible light,

a photovoltaically-active region of the flexible power source having an area of 5-50 mm², and

at least one photovoltaically-active site in the photovoltaically-active region of the flexible power source being disposed 0.4-3 mm from a nearest one of the plurality of photosensors to the site.

There is further provided in accordance with some applications of the present invention, intraocular apparatus configured to be implanted entirely in a subject's eye, the intraocular apparatus including:
a power source, configured to power the intraocular apparatus, the power source including:
   (i) at least one energy receiver configured to receive non-visible light through the lens of the eye and to extract power from the non-visible light; and
   (ii) at least one power storage element, configured to power the intraocular apparatus when the intraocular apparatus is not receiving energy from outside of the eye;
a plurality of stimulating electrodes;
a photosensor array including a plurality of photosensors, each photosensor configured to detect photons and to generate a signal in response thereto; and
driving circuitry, coupled to the power source and to the photosensors, and configured to receive the signals from the photosensors and to utilize the voltage drop to drive the electrodes to apply currents to a retina of the eye in response to the signals from the photosensors.

The present invention will be more fully understood from the following detailed description of applications thereof, taken together with the drawings, in which:

### BRIEF DESCRIPTION OF THE DRAWINGS

FIG. 1 is a schematic illustration of intraocular apparatus implanted in an eye of a subject, in accordance with some applications of the present invention;

FIGS. 2A and 2B are schematic illustrations of top and side views respectively of intraocular apparatus for implantation in an eye of a subject, in accordance with some applications of the present invention;

FIG. 3 is a schematic illustration of intraocular apparatus implanted in an eye of a subject, in accordance with some applications of the present invention;

FIG. 4 is a schematic illustration of an additional configuration of intraocular apparatus implanted in an eye of a subject, in accordance with some applications of the present invention;

FIG. 5 is a schematic illustration of intraocular apparatus implanted in an eye of a subject, in accordance with some applications of the present invention; and

FIG. 6 is a schematic illustration of an example of a power source of the intraocular apparatus, in accordance with some applications of the present invention.

### DETAILED DESCRIPTION OF EMBODIMENTS

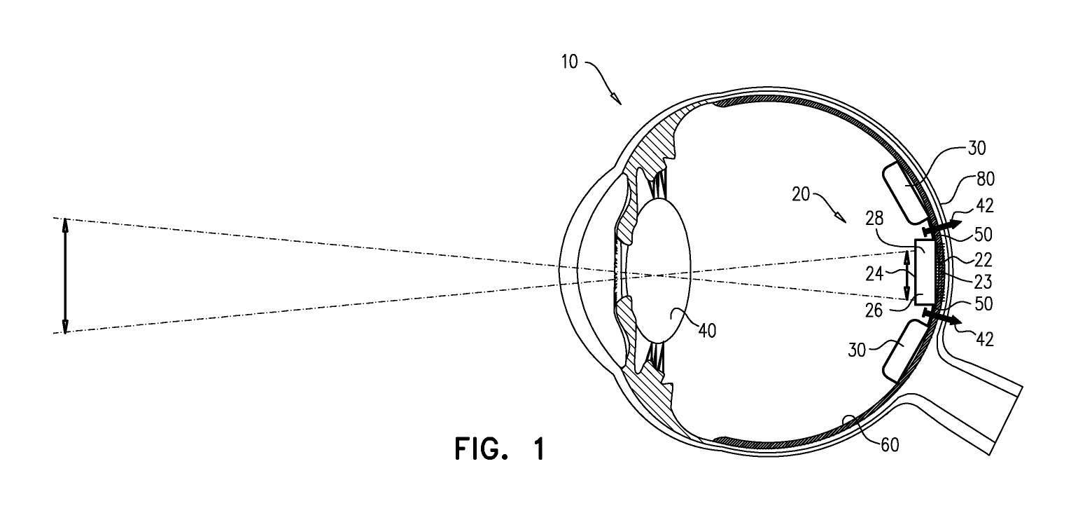

Reference is first made to FIG. 1, which is a schematic illustration of intraocular apparatus 20 implanted in an eye 10 of a subject, in accordance with some applications of the present invention. As shown, apparatus 20 is separated into a plurality of modules which are electrically and mechanically coupled and which are implanted as a single unit onto retina 60 to together form intraocular apparatus 20. Typically, apparatus 20 comprises a retinal prosthesis module 28 comprising a photosensor array 24 which comprises a plurality of photosensors and which receives an ambient image through lens 40 of eye 10. Typically, lens 40 is a native or a prosthetic lens. Apparatus 20 further comprises a power source module 30 configured to power apparatus 20, and a flexible electrical connector 50 mechanically and electrically connecting photosensor array 24 to power source 30. Typically, flexible electrical connector 50 has a length of at least 0.4 mm and/or less than 3 mm, and typically comprises thinned silicon. As shown in FIG. 1, for some applications, apparatus 20 comprises more than one, e.g., first and second, power sources modules 30, positioned on either side of retinal prosthesis module 28. In such cases, flexible electrical connector 50 comprises first and second flexible electrical connectors 50 connecting both power sources modules 30 to retinal prosthesis module 28. Typically, retinal prosthesis module 28, power source module 30 and flexible electrical connector 50 are formed along a single piece of thinned silicon.

Typically, retinal prosthesis module 28 further comprises an electrode array 22 comprising stimulating micro-electrodes 23. Retinal prosthesis module 28 additionally comprises driving circuitry 26 (typically including image processing electronics) which is coupled to power source module 30 and to photosensor array 24. Driving circuitry 26 receives power from power source 30 to drive electrodes 23 to apply currents to retina 60 in response to signals from photosensor array 24 in retinal prosthesis module 28, in order to stimulate retina 60.

Typically, implanting intraocular apparatus 20 as a plurality of electrically and mechanically connected modules as shown in FIG. 1, reduces mechanical stress on retina 60 during motion of eye 10. This is in contrast to implanting onto the retina a single, high-volume unit which may apply mechanical stress to the retina leading to retinal detachment or separation of the retinal implant from the retina. Additionally, the natural curvature of retina 60 may make it difficult to implant a single, large-area unit onto the retina. Apparatus 20, being divided into separate modules which are electrically and mechanically connected by flexible connectors 50, is typically flexible and can bend in order to conform to the natural curvature of retina 60, facilitating proper implantation and attachment of apparatus 20.

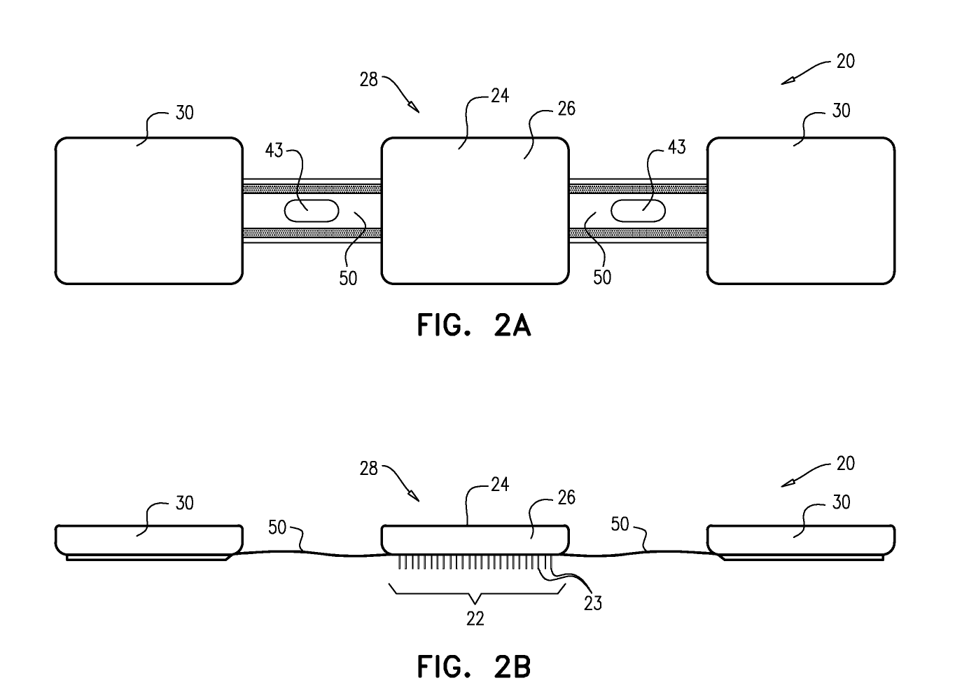

Reference is now made to FIGS. 2A and 2B, which are schematic illustrations of top (FIG. 2A) and side (FIG. 2B) views of apparatus 20, in accordance with some applications of the present invention. FIGS. 2A and 2B show apparatus 20 prior to implantation in eye 10 and prior to bending of flexible connectors 50 for facilitating implantation and conforming to the natural curvature of the eye. As shown in FIGS. 2A-B, power source modules 30 are positioned on either side of retinal prosthesis module 28 and are mechanically and electrically connected to retinal prosthesis module 28 by flexible connectors 50. Typically, flexible connector 50 comprises thinned silicon, and retinal prosthesis module 28, power source module 30 and flexible electrical connector 50 are formed along a single piece of thinned silicon. The thinned silicon is typically durable and bio-compatible. Additionally, the thinned silicon functions as an electrical conducting material typically without additional use of a conductive metallic layer, thus enhancing the bio-compatibly of connector 50. Flexible connector 50 is typically fabricated to an ultra-thin conductive silicon strip (e.g., 20-50 microns thick) by conventional fabrication processes known in the art. For some applications, techniques described in U.S. Pat. No. 7,560,802, which is incorporated herein by reference are practiced in combination with techniques and apparatus described herein, with regard to thinned silicon and implementation of a conducting silicon strip.

Reference is again made to FIG. 1 and FIGS. 2A-B. Typically, apparatus 20 is implanted onto retina 60 and anchored to a sclera 80 of eye 10 using one or more anchoring elements 42, e.g., anchoring tacks. As shown in FIG. 2A flexible connector 50 is shaped to define at least one, e.g., two, holes 43 shaped and sized to receive anchoring elements 42 therethrough. When apparatus 20 is implanted onto retina 60, anchoring elements 42 are positioned in holes 43 and penetrate sclera 80 to secure apparatus 20 to eye 10. Positioning anchoring elements 42 in holes 43 between generally rigid power sources 30, and pressing flexible connector 50 to retina 60, typically causes the bending of apparatus 20 and conforming of apparatus 20 to the natural curvature of retina 60. It is noted that apparatus 20 may be secured to eye 10 by using other anchoring mechanisms.

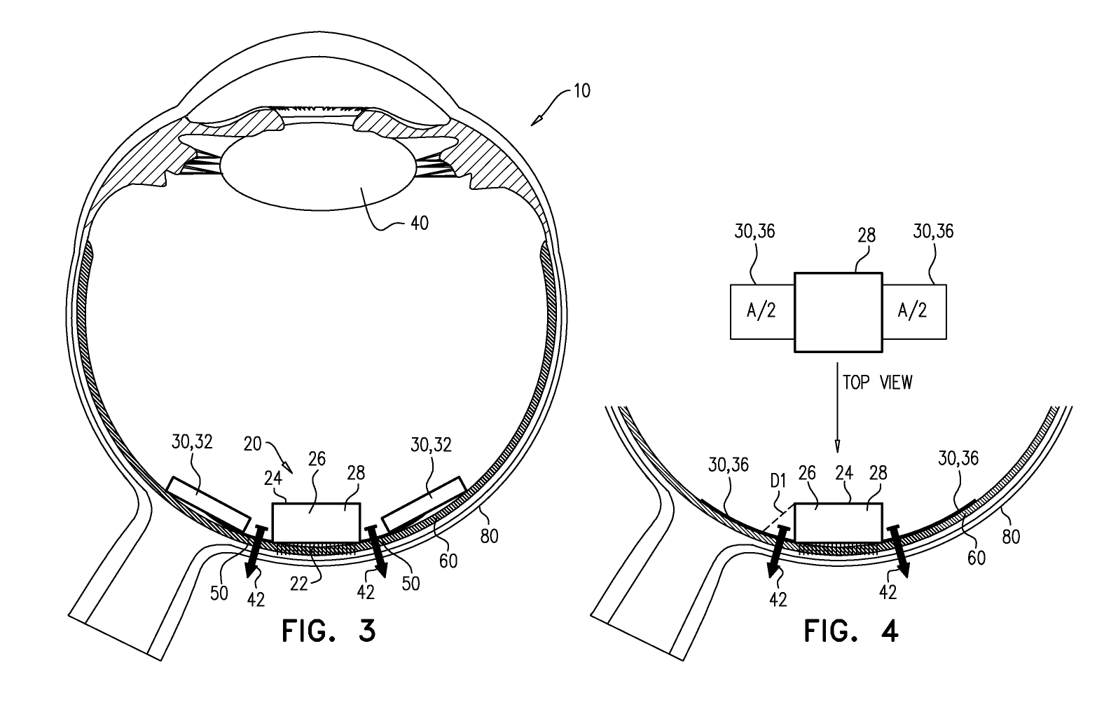

Reference is now made to FIG. 3, which is a schematic illustration of intraocular apparatus 20 implanted in eye 10 of the subject, in accordance with some applications of the present invention. As provided by some applications, power source module 30 comprises energy receiver 32. For some applications, energy receiver 32 comprises photovoltaic cells configured to receive light and convert the optical energy from the light into electrical energy for powering apparatus 20. Energy receiver 32 receives non-visible light, e.g., light outside of 390-700 nm, typically infrared light, through lens 40 of eye 10 and extracts power from the non-visible light to power apparatus 20. For some applications, energy receiver 32 receives non-visible light from an extraocular device (not shown) comprising a light source, e.g., a laser or an LED, which emits the non-visible light, e.g., infrared light, toward eye 10. For some applications, techniques described in U.S. Pat. No. 8,150,526, which is incorporated herein by reference, are practiced in combination with techniques and apparatus described herein, with regard to an extraocular device comprising a light source. Additionally or alternatively, energy receiver 32 is configured to extract power from ambient visible and/or non-visible light in a highly lit environment, and does not rely on light from an extraocular laser or other dedicated component of apparatus provided to power intraocular apparatus 20. As shown, energy receivers 32 are positioned on retina 60 facing the iris of eye 10, for absorbing the light.

As shown in FIG. 3, apparatus 20 comprises two energy receivers 32 positioned on either side of retinal prosthesis module 28, each energy receiver 32 being connected electrically and mechanically to module 28 by flexible connector 50. Typically, power from energy receivers 32 is conducted through flexible connector 50 to retinal prosthesis module 28 to drive driving circuitry 26 to drive electrodes 23 to apply currents to retina 60 in response to signals from photosensor array 24 of retinal prosthesis module 28, in order to stimulate retina 60.

Typically, by partitioning apparatus 20 into several relatively small connected units and having two energy receivers 32, rather than one larger energy receiver, the light absorbing area of apparatus 20 is increased without unduly increasing the size of retinal prosthesis module 28, thereby enhancing light reception by apparatus 20 and also facilitating widening the scanning angle with respect to the center of the iris.

Reference is now made to FIG. 4, which is a schematic illustration of an additional configuration of intraocular apparatus 20 implanted in eye 10 of the subject, in accordance with some applications of the present invention. For some applications, power source 30 comprises flexible power source 36 comprising thinned silicon (similar to flexible connector 50) and coupled to retinal prosthesis module 28 and configured to power apparatus 20. Flexible power source 36 typically comprises a photovoltaic energy receiver (the thinned silicon has PN junctions on an upper surface thereof which act as photovoltaic cells) configured to receive non-visible light and to extract power from the non-visible light, similar to energy receiver 32 described hereinabove with reference to FIG. 3. Typically, flexible power source 36 has a photovoltaically-active region having an area A of 5-50 mm². Additionally, at least one photovoltaically-active site in the photovoltaically-active region of flexible power source 36 is disposed at a distance D1 which is at least 0.4 mm and/or less than 3 mm from a nearest one of the plurality of photosensors in photosensor array 24 to the site. Typically, flexibility of power source 36 enables spreading power source 36 over a relatively large area of retina 60, enabling the capturing of a relatively large amount of light and converting it to electrical power for powering apparatus 20. It is noted that although flexible power source 36 is shown in FIG. 4 as being on both sides of retinal prosthesis module 28, the scope of the present invention includes having flexible power source 36 on only one side of retinal prosthesis module 28.

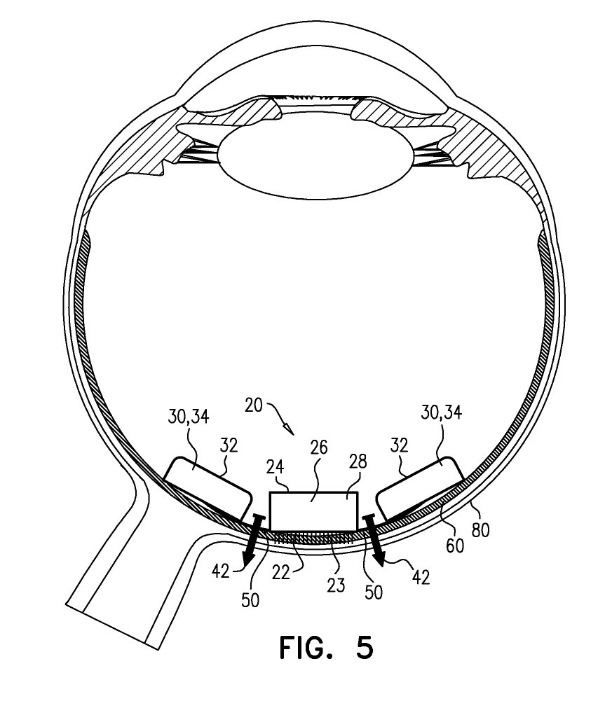

Reference is now made to FIG. 5, which is a schematic illustration of intraocular apparatus 20 implanted in eye 10 of the subject, in accordance with some applications of the present invention. For some applications, power source module 30 comprises at least one power storage element 34. Power storage element 34 typically stores power to provide power to apparatus 20 when apparatus 20 is not receiving energy from outside of the eye. Typically, power storage element 34 comprises a battery, and/or a high value capacitor, and/or a supercapacitor.

As noted hereinabove, for some applications, power storage element 34 comprises a rechargeable battery configured to store sufficient charge for operation of apparatus 20 for 1-10 hours, e.g., 1-4 hours. Typically the rechargeable battery has a total charge of greater than 0.1 mAh and/or less than 10 mAh, e.g. in the range of 0.1 mAh-10 mAh.

For some applications, power storage element 34 comprises a plurality of miniature batteries, e.g., solid state batteries. Typically, power storage element 34 comprises a plurality of stacked dies, each die comprising a plurality of miniature batteries integrated into the die for gaining larger charge capacity for generally the same area.

Typically, power storage element 34 is charged by energy-receiving photovoltaic cells in energy receiver 32. For such applications, the photovoltaic cells are placed inside power storage element 34, typically on a surface of power storage element 34 facing the iris of eye 10. When eye 10 is illuminated, the light is received by the photovoltaic cells, and power is extracted from the light and conducted to power storage element 34 where the power is stored. For some applications, a diode, e.g., a Schottky diode, is used to ensure that the photovoltaic cells only drive current into power storage element 34, but do not consume current from power storage element 34. Alternatively, a rectification circuit, e.g., implemented in a CMOS ASIC, is used to inhibit the photovoltaic cells from applying a load to the retinal prosthesis module 28.

As shown in FIG. 5, apparatus 20 comprises two power storage elements 34, positioned on either side of retinal prosthesis module 28, each power storage element 34 being connected electrically and mechanically to module 28 by flexible connector 50. Typically, power from power storage element 34 is conducted through flexible connector 50 to retinal prosthesis module 28 to drive driving circuitry 26 to drive electrodes 23 to apply current pulses to retina 60 in response to signals from photosensor array 24 of retinal prosthesis module 28, in order to stimulate retina 60.

Typically, when power is stored in power storage element 34, apparatus 20 can be powered also when apparatus 20 is not receiving light from outside of the eye. Therefore, it is not necessary to continuously illuminate apparatus 20. Typically, in cases in which apparatus 20 does not comprise a power storage element 34, in order to provide continuous power to apparatus 20, an extraocular light source, e.g., an infra-red laser is provided. Typically the laser is mounted on a pair of eyeglasses worn by the subject and is positioned such that light emitted by the laser is received by apparatus 20. Use of power storage element 34 may allow the subject to illuminate the eye only when charge in power storage element 34 is low, without the need for continuous wearing of glasses and for continuous illumination of the eye by an extraocular light source. Thus, apparatus 20 is not continuously dependent on an extraocular light source for providing power. In other words, when sufficient energy is stored inside apparatus 20 (e.g., inside storage element 34), there is generally no need for illumination of the eye by a dedicated extraocular light source mounted on glasses. Instead, the eye is illuminated by a charging light source that is held in front of the eye for a typically short amount of time which is sufficient to charge storage element 34. Thereby, the subject is offered the option of not wearing special-purpose glasses (for example, the subject may wear any type of glasses for esthetic or corrective reasons).

Additionally or alternatively, a scanning angle of eye 10 is not limited by the size of the light beam from the extraocular light source. That is, when the subject gazes to the right or left and an extraocular power source is therefore not illuminating energy receiver sufficient power remains in power storage element 34 for intraocular apparatus 20 to continue to operate.

Additionally or alternatively, due to use of power storage element 34, implantation of some or all components of apparatus 20 (e.g., power storage element 34 in particular), is not limited to a particular area of the retina, e.g., the fovea. Thus, for some applications, the scope of the present invention includes implanting more than one apparatus 20 in a single procedure in a single eye, or subsequently implanting a second apparatus 20, e.g., when a first, previously-implanted, apparatus 20 has ended its lifetime or is superseded by a newer version of apparatus 20.

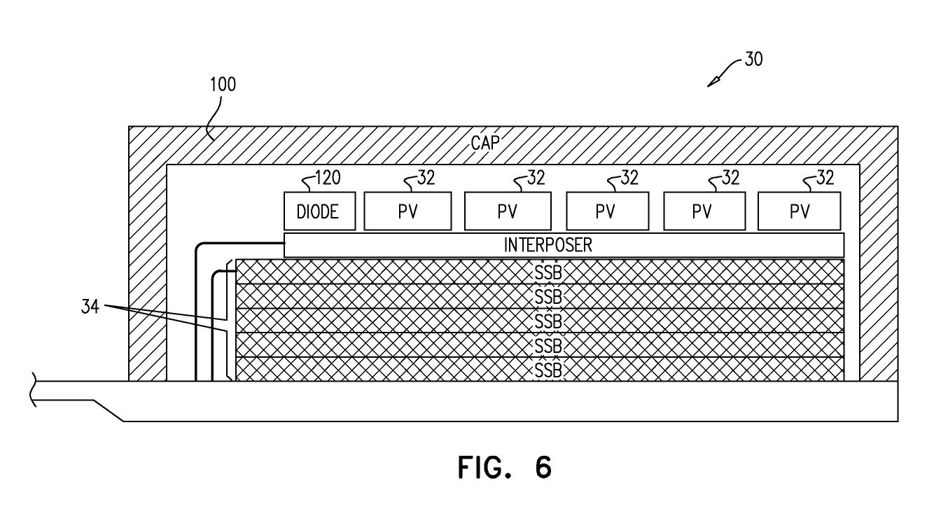

Reference is now made to FIG. 6, which is a schematic illustration of power source 30 of intraocular apparatus 20, in accordance with some applications of the present invention. As shown in FIG. 6, power source 30 comprises power storage element 34 comprising a plurality of stacked dies each die comprising miniature batteries, e.g., solid state batteries (SSB), integrated into the die. Power source 30 additionally comprises energy receiver 32 comprising a plurality of photovoltaic cells placed on an interposer that connects the photovoltaic cells. As shown, photovoltaic cells are placed on a top side of the power storage element. A transparent cap 100 allows passage of light therethrough toward the photovoltaic cells of power receiver 32. Typically, a diode 120, e.g., a Schottky diode, is used to ensure that the photovoltaic cells only drive current into power storage element 34, but do not consume current from power storage element 34.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Intraocular apparatus configured to be implanted entirely in a subject's eye, the intraocular apparatus comprising:
a photosensor array comprising a plurality of photosensors configured to receive an ambient image;
a power source, configured to power the intraocular apparatus; and
a flexible 0.4-3 mm electrical connector, connecting the photosensor array to the power source.

2. The intraocular apparatus according to claim 1, wherein the flexible connector comprises thinned silicon.

3. The intraocular apparatus according to claim 1, wherein the photosensor array, the power source and the flexible electrical connector are formed along a single piece of thinned silicon.

4. The intraocular apparatus according to claim 1, wherein the power source comprises at least one power storage element, configured to power the intraocular apparatus when the intraocular apparatus is not receiving energy from outside of the eye.

5. The intraocular apparatus according to claim 4, wherein the at least one power storage element comprises a rechargeable battery configured to store sufficient charge for operation of the intraocular apparatus for 1-10 hours.

6. The intraocular apparatus according to claim 5, wherein the rechargeable battery has a total charge of 0.1-10 mAh.

7. The intraocular apparatus according to claim 5, wherein the at least one power storage element comprises a plurality of stacked dies, each die comprising a plurality of miniature batteries integrated into the die.

8. The intraocular apparatus according to claim 4, wherein:
the at least one power storage element comprises first and second power storage elements, positioned on either side of the photosensor array,
the flexible electrical connector is a first flexible electrical connector and connects the first power storage element to the photosensor array, and
the intraocular apparatus further comprises a second flexible electrical connector, which connects the second power storage element to the photosensor array.

9. The intraocular apparatus according to claim 1, wherein the power source comprises at least one energy receiver configured to receive non-visible light through the lens of the eye and to extract power from the non-visible light.

10. The intraocular apparatus according to claim 9, wherein the at least one energy receiver is configured to receive light with wavelength that is outside of 390-700 nm.

11. The intraocular apparatus according to claim 9, wherein:
the at least one energy receiver comprises first and second energy receivers, positioned on either side of the photosensor array,
the flexible electrical connector is a first flexible electrical connector and connects the first energy receiver to the photosensor array, and
the intraocular apparatus further comprises a second flexible electrical connector, which connects the second energy receiver to the photosensor array.

12. The intraocular apparatus according to claim 9, wherein the power source further comprises at least one power storage element, configured to power the intraocular apparatus when the intraocular apparatus is not receiving energy from outside of the eye.

13. The intraocular apparatus according to claim 12, further comprising an extraocular device comprising a light source configured to emit the non-visible light toward the eye, wherein the at least one power storage element is configured to store power extracted from the non-visible light for at least one hour, and wherein the intraocular apparatus is configured to use the stored power to power the intraocular apparatus.

14. The intraocular apparatus according to claim 13, wherein the light source comprises a laser.

15. The intraocular apparatus according to claim 12, wherein the at least one power storage element comprises a rechargeable battery configured to receive the power extracted by the energy receiver.

16. The intraocular apparatus according to claim 1, wherein the intraocular apparatus further comprises (i) an electrode array comprising electrodes, and (ii) driving circuitry coupled to the power source and to the photosensor array, and configured to receive power from the power source to drive the electrodes to apply currents to the retina in response to signals from the plurality of photosensors in the photosensor array.

17. Intraocular apparatus configured to be implanted entirely in a subject's eye, the intraocular apparatus comprising:
a photosensor array comprising a plurality of photosensors configured to receive an ambient image;
a power source, configured to power the intraocular apparatus; and
a flexible electrical connector, connecting the photosensor array to the power source,
the photosensor array, the power source and the flexible electrical connector being formed along a single piece of thinned silicon.

18. Intraocular apparatus configured to be implanted entirely in a subject's eye, the intraocular apparatus comprising:
a photosensor array comprising a plurality of photosensors, configured to receive an ambient image; and
a flexible power source comprising thinned silicon and coupled to the plurality of photosensors, configured to power the intraocular apparatus and comprising a photovoltaic energy receiver configured to receive non-visible light and to extract power from the non-visible light,
a photovoltaically-active region of the flexible power source having an area of 5-50 mm², and
at least one photovoltaically-active site in the photovoltaically-active region being disposed 0.4-3 mm from a nearest one of the plurality of photosensors to the site.

19. Intraocular apparatus configured to be implanted entirely in a subject's eye, the intraocular apparatus comprising:
a power source, configured to power the intraocular apparatus, the power source comprising:
(i) at least one energy receiver configured to receive non-visible light through the lens of the eye and to extract power from the non-visible light; and
(ii) at least one power storage element, configured to power the intraocular apparatus when the intraocular apparatus is not receiving energy from outside of the eye;
a plurality of stimulating electrodes;
a photosensor array comprising a plurality of photosensors, each photosensor configured to detect photons and to generate a signal in response thereto; and
driving circuitry, coupled to the power source and to the photosensors, and configured to receive the signals from the photosensors and to utilize the voltage drop to drive the electrodes to apply currents to a retina of the eye in response to the signals from the photosensors.
